(19) United States
(12) Patent Application Publication
(10) Pub. No.: US 2007/0191633 A1
(43) Pub. Date: Aug. 16, 2007

(54) Mixed anionic surfactant composition for oil recovery

(75) Inventors: Paul Daniel Berger, Sugar Land, TX (US); Christie Huimin Berger, Sugar Land, TX (US)

Correspondence Address: PAUL D. BERGER;c/o OCT, 12822 Park One Drive, Sugar Land, TX, 77478, US

(73) Assignee: CHRISTIE HUIMIN BERGER, SUGAR LAND, TX (US)

(21) Appl. No.: 11/706,614

(22) Filed: Feb. 15, 2007

Related U.S. Application Data
(60) Provisional application No. 60/773,398, filed on Feb. 15, 2006.

(51) Int. Cl. C07C 309/00

(52) U.S. Cl. 562/41

(57) ABSTRACT

A composition for recovering oil from subterranean formation by injecting an aqueous fluid containing from about 0.05 to about 2.0% by weight of a bi-functional surfactant or a mixture of surfactants containing one or more of the following structures;

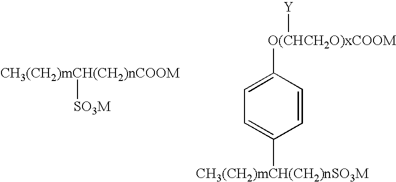

Optionally the aqueous fluid may contain mixtures of individual surfactants having carboxylic, and sulfonate or sulfate functionalities. The remainder of the composition includes water or brine, a cosolvent and optionally a viscosity control agent, and optionally an alkali.

### CROSS REFERENCE TO RELATED APPLICATIONS

This application is based on provisional application Ser. No. 60/773,398 filed on Feb. 16, 2006.

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

The present invention relates to a composition for the recovery of oil from subterranean oil bearing reservoirs, and more particularly the present invention is to improved oil recovery operations involving the injection into reservoirs of a composition containing mixtures of surfactants containing a weak anionic functionality group and a strong anionic functionality group.

In the recovery of oil from subterranean reservoirs it usually is possible to recover approximately 15%-20% of the original oil in place by primary recovery. Secondary recovery methods such as well stimulation or water flooding are applied after the amount of oil recovered by primary recovery becomes uneconomical. Secondary recovery methods can recover approximately an additional 15%-30% of the original oil in place which leaves the reminder of the oil unrecoverable unless other means such as tertiary recovery processes are applied. These tertiary recovery methods include but are not limited to the use of miscible and immiscible gases and liquids, steam, foam, alkali, surfactants, and polymers.

It has been known that many factors including but not limited to the interfacial tension between the injection brine and the residual oil, the relative mobility of the injected brine, and the wettability characteristics of the rock surfaces comprising the reservoir are all important in determining the amount of oil recovered by tertiary recovery. Numerous studies have found that the addition of surfactants to the injection brine can alter the interfacial and wetting properties to help overcome the high capillary pressure and increase the oil recovery. In many cases the addition of a polymer along with the surfactant or immediately after the surfactant can increase the mobility ratio between the injected brine and oil thus further improving the sweep efficiency of the flood.

Because the injection brine composition varies, it is important to use the brine available at the injection site for the tertiary process in order to be economically feasible. It is important to have surfactants that are compatible with brines having wide ranges of total dissolved solids (TDS) and divalent cations such as those of calcium and magnesium. The problem with many of the presently used surfactants in tertiary oil recovery is that they are incompatible with the brines containing high TDS and divalent cations that are often found at the injection site. Costly water treatment processes or using an alternate fresh water source makes the tertiary recovery process economically unfeasible in many cases. Therefore it is important to have surfactants that are tolerant to the high TDS and divalent cations.

### BRIEF SUMMARY OF THE INVENTION

The present invention is directed to the composition of surfactants containing a weak anionic functionality group and a strong anionic functionality group, which composition is formulated into a concentrated surfactant blend containing a aqueous solvent such as water or brine, and a co-surfactant/solvent such as a lower molecular weight alcohol or alcohol ether. The concentrated surfactant blend is added in a concentration range of about 0.05% to about 5% to the injection brine and introduced into the subterranean hydrocarbon containing formation by (a) injecting into said formation through one or more injection wells, and (b) displacing said solution into the formation to recover hydrocarbons from one or more production wells. The injection and producing wells may be the same or different. Depending on the reservoir conditions, other additives may be added to the injection brine including strong or weak alkalis, viscosifiers, corrosion and scale inhibitors, and others known to those familiar with the art.

In the present invention, the weak and strong anionic functionality groups may be combined into the same molecule as a bi-functional surfactant, or be present as mixtures of surfactants containing one or more strong anionic functionality group and one or more weak anionic functionality group.

Non-exclusive examples where the weak and strong functionality groups are included in the same molecule as bi-functional surfactant are:

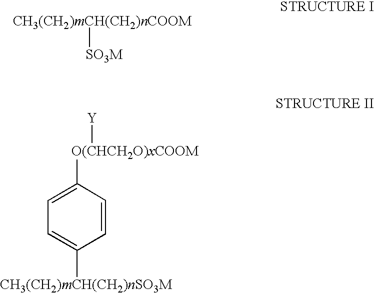

where m+n=5-28

### M=H, Na, K, NH₃, Amine, Ca, Mg,

### Y=H, CH₃, or CH₃CH₂,

and x=0-30 or more

The product described in Structure I is made by sulfonating an unsaturated fatty acid using any of a number of sulfonation processes known to those familiar with the art including, but not limited to, SO₃ thin film sulfonation, oleum, chlorosulfonic acid, cold SO₂/SO₃ and sulfamic acid. The product described in Structure II is made by reacting the carboxylated phenol with sulfonic acid obtained from the sulfonation of an olefin using the procedure described in U.S. Pat. No. 6,043,391. The phenol moiety may contain from about 0 to about 30 or more moles of an alkoxy group such as ethylene oxide (EO), propylene oxide (PO), or mixtures of EO and PO, or sequences of EO and PO, to adjust the solubility and molecular weight of the surfactant.

Non-exclusive examples of the mixtures of surfactants containing one or more strong anionic functionality group and one or more weak anionic functionality group are: linear alkoxylated alcohol carboxylates, branched alkoxylated alcohol carboxylates, carboxylated aryl alkoxylates, linear alkyl aryl sulfonates, branched alkyl aryl sulfonates, aryl linear alkyl sulfonates, aryl branched alkyl sulfonates, linear alkyl ether sulfates, branched alkyl ether sulfates, linear alkyl ether sulfonates, branched alkyl ether sulfonates, linear alkyl sulfates, branched alkyl sulfates, linear alkyl sulfonates, branched alkyl sulfonates, and their salts including, Na, K, NH₃, Amine, Ca, Mg.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The present invention is directed to the composition of salt and divalent cations tolerant surfactants containing a weak anionic functionality group and a strong anionic functionality group for recovering crude oil from subterranean hydrocarbon formation. The weak and strong anionic functionality groups may be combined into the same molecule as one or more bi-functional surfactants or mixtures of surfactants containing one or more strong anionic functionality group and one or more weak anionic functionality group may be used.

Non-exclusive examples of the weak and strong functionality groups are included in the same molecule as bi-functional surfactants are:

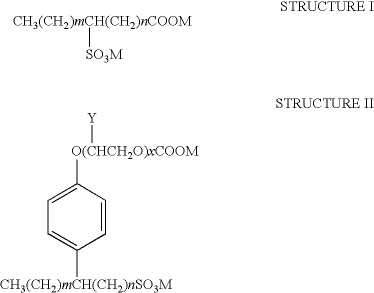

where m+n=5-28

### M=H, Na, K, NH₃, Amine, Ca, Mg,

### Y═H, CH₃, or CH₃CH₂,

and x=0-30 or more

The product described in Structure I is made by sulfonating an unsaturated fatty acid using any of a number of sulfonation processes known to those familiar with the art including but not limited to SO₃ thin film sulfonation, oleum, chlorosulfonic acid, cold SO₂/SO₃ and sulfamic acid. The product described in Structure II is made by reacting a carboxylated phenol with the sulfonic acid obtained from the sulfonation of an olefin as described in U.S. Pat. No. 6,043,391. The phenol moiety may contain from about 0 to about 30 or more moles of an alkoxy group such as EO PO, or mixtures of EO and PO, or sequences of EO and PO, to adjust the solubility and molecular weight of the surfactant.

Non-exclusive examples of the mixtures of surfactants containing one or more weak anionic functionality groups and one or more strong anionic functionality groups are mixtures of linear alkoxylated alcohol carboxylates, branched alkoxylated alcohol carboxylates, carboxylated arylalkoxylates, with linear alkyl aryl sulfonates, branched alkyl aryl sulfonates, aryl linear alkyl sulfonates, aryl branched alkyl sulfonates, linear alkyl ether sulfates, branched alkyl ether sulfates, linear alkyl ether sulfonates, branched alkyl ether sulfonates, linear alkyl sulfates, branched alkyl sulfates, linear alkyl sulfonates, branched alkyl sulfonates, and their salts including, Na, K, NH₃, Amine, Ca, Mg.

Specific, but not limiting examples, are mixtures of sodium C1416 alpha-olefin sulfonate (AOS) with branched sodium tridecyl-6 EO carboxylate. Another example is a blend of linear sodium alkyl benzene sulfonate, branched sodium alkyl benzene sulfonate and sodium nonylphenol-10 EO carboxylate.

The ratio of the weak to strong anionic functionality groups is determined by the characteristics of the crude oil, the brine, temperature and the type of formation in which they are to be used. The final composition is formulated so that it is compatible with the brine and enhances the recovery of oil under the bottom hole conditions.

The present invention of surfactants containing a weak anionic functionality group and a strong anionic functionality group are usually formulated into a concentrated surfactant blend containing a aqueous solvent such as water or brine, and a co-surfactant/solvent such as a lower molecular weight alcohol or alcohol ether. Non-exclusive examples of the co-surfactant/solvent are iso-propanol, n-butanol, and ethylene glycol monobutyl ether. The concentrated surfactant solution is added in a concentration range of about 0.05% to about 5% to the brine and introduced into the subterranean hydrocarbon containing formation by (a) injecting into said formation through one or more injection wells, and (b) displacing said solution into the formation to recover hydrocarbons from one or more production wells. The injection and producing wells may be the same or different. Depending on the reservoir conditions, other additives may be added to the injection brine including strong or weak alkalis, viscosifiers, corrosion and scale inhibitors, and others known to those familiar with the art.

The concentrated surfactant blend containing strong and weak anionic functionality groups are compatible and can be added to a wide range of brines containing different amount of total dissolved solids and multivalent cations such as Ca⁺² and Mg⁺² and injected into the oil bearing reservoirs to recover residual oil. They can also be injected with gas, such as N₂ or CO₂.

In accordance with a preferred embodiment of the invention, there is disclosed a composition for improving the recovery of oil containing
a) one or more bi-functional surfactants containing both a weak and a strong anionic functionality groups on one molecule, or a mixture of a least one or more surfactant containing a strong anionic functionality group and at least one or more surfactants containing a weak anionic functionality group,
b) an aqueous based solvent,
c) optionally one or more co-surfactants/solvents,
d) optionally a viscosity improving agent, and
e) optionally an alkali.

The preferred strong anionic functionality is derived from a sulfate or sulfonate group and the preferred weak anionic functionality is derived from a carboxylate group. The aqueous solvent may be water or a synthetic brine or a brine that is produced from the reservoir containing various mono and divalent ion salts including but not limited to the sulfonate, chloride, carbonate and/or bicarbonate salts of sodium, potassium, calcium, magnesium, strontium, barium, and/or iron originally present or intentionally added to the aqueous solvent. The co-surfactant/solvent includes but is not limited to a short chained alcohol, glycol, or ether such as methanol, ethanol, propanol, Isopropanol, butanol, iso-butanol, glycerin, ethylene glycol, propylene glycol, ethylene glycol monobutyl ether. The alkali includes but is not limited to sodium hydroxide or sodium carbonate. The viscosity improving agent may includes but is not limited to any of a number of polymers known to those familiar with the art including polyacrylamide, xanthan gum, and block polymers of acrylamide and Copolymer of acrylic acid and 2-Acrylamido-2-Methylpropyl Sulfonic Acid.

An objective of the present invention is to provide a surfactant composition to improved oil recovery that is effective over a wide range of electrolyte and divalent anion concentrations.

Another objective of the present invention is to provide a surfactant composition to improved oil recovery with minimum adsorption onto the formation.

Other objectives and advantages of the present invention will become apparent from the following descriptions, taken in connection with the accompanying drawings, wherein, by way of illustration and example, an embodiment of the present invention is disclosed.

Following are examples illustrating the utility of the present invention for application in the recovery of oil from subterranean reservoirs. The Interfacial tension (IFT) between the crude oil/injection brine using the composition of the present invention is used to illustrate the efficiency of the present invention. It is well documented that after primary oil recovery and secondary oil recovery the Capillary Number is about 10⁻⁶. See for instance Basic Concepts in Enhanced Oil Recover Processes, p 18-19, 90. The capillary number is defined as:

Nc=μV/σ

where

### Nc=Capillary Number

μ=displacing fluid viscosity

### V=interstitial velocity

### σ=IFT between the displacing fluid and the crude oil.

Increasing the Capillary Number to a value above 10⁻³ has been shown to result in a substantial increase in the recovery of trapped oil after waterflooding has become ineffective (Basic Concepts in Enhanced Oil Recover Processes, p 108). The viscosity and the velocity cannot be increased substantially without damaging the reservoir, however, the IFT can easily be reduced 3 to 4 orders of magnitude by the proper choice of surfactant. Thus a surfactant can reduce the IFT between an oil and an aqueous medium from 3-30 mN/m to less than 10⁻² mN/m resulting in an increase in the capillary number to grater than 10⁻² and improve the oil recovery.

In the following examples, IFT has been used as a measure of the suitability of a particular surfactant as a candidate for enhanced oil recovery.

Table I lists the surfactants used to in the examples chosen to demonstrate the utility and novelty of the invention. In all cases the surfactant formulation consisted of 50% by weight surfactant, 25% by weight Ethylene glycol Monobutyl ether (co-surfactant/solvent), and 25% by weight water. Also in all cases the surfactant formulation was added to the injection brine at a concentration of 0.10 weight percent.

TABLE I
Surfactants Used In Examples

| SURFACTANT | CHEMICAL DESCRIPTION | FUNCTIONALITY DESCRIPTION |
|---|---|---|
| A | Sodium salt of linear dodecyl benzene | Surfactant with strong anionic |
| | sulfonate EW = 342 | functionality group |
| B | Sodium salt of linear dodecyl benzene | Surfactant with strong anionic |
| | sulfonate EW = 405 | functionality group |
| C | Sodium salt of nonylphenol + 10 EO | Surfactant with weak anionic |
| | carboxylate | functionality group |
| D | Sodium salt of sulfonated erucic acid | Surfactant with both weak and strong |
| | (structure I where M = Na, m + n = 19) | anionic functionality groups |
| E | Sodium salt of sulfonated oleic acid (structure | Surfactant with both weak and strong |
| | I where M = Na, m + n = 15) | anionic functionality groups |
| F | Sodium salt of alkoxylated alkylphenol | Surfactant with both weak and strong |
| | sulfonate (structure II where M = Na, m + n = 12, | anionic functionality groups |
| | and x = 10) | |

Table II is the brine compositions that were used for the IFT testing to show the effect of total dissolved solids and divalent ion concentration on the IFT obtained using various surfactants.

TABLE II
Brine Compositions

| BRINE | 1 | 2 | 3 |
|---|---|---|---|
| NaCl, % | 1.0 | 7.0 | 10.0 |
| CaCl2—2H2O, % | 0.1 | 0.1 | 1.5 |
| MgCl2—6H2O, % | 0.1 | 0.1 | 1.5 |

Table III compares the IFT obtained with 0.10% by weight of various surfactant concentrates in the three brines against a crude oil having 34 API Gravity at 42° C. All IFTs were obtained using a University of Texas Model 500 spinning drop interfacial tensiometer after spinning at 42° C. for 1 hour.

TABLE III
Comparison of IFTs With Various Surfactants
Oil API Gravity = 34
Testing Temperature = 42° C.

| TEST | SURFACTANTS | BRINE 1 | BRINE 2 | BRINE 3 |
|---|---|---|---|---|
| 1 | A | 0.225 | Precipitate | Precipitate |
| 2 | B | 0.177 | Precipitate | Precipitate |
| 3 | C | 0.077 | 0.092 | 0.191 |
| 4 | D | 0.0097 | 0.0072 | 0.0075 |
| 5 | E | 0.0086 | 0.0012 | 0.0016 |
| 6 | 1:1 A + C | 0.0029 | 0.0057 | 0 0.0076 |
| 7 | 1:1 B + C | 0.0054 | 0.0063 | 0.00089 |
| 8 | 1:1:1A + B + C | 0.0087 | 0.0021 | 0.0023 |
| 9 | 1:1 D + E | 0.0045 | 0.0051 | 0.0051 |
| 10 | F | 0.0022 | 0.0023 | 0.00298 |

The data obtained from Table III shows that surfactant A and B are soluble in the lower salt concentration Brine 1, yet the IFTs are too high to effectively remove the residual oil. Furthermore, surfactants A and B are not salt tolerant and precipitated in Brines 2 and 3. Surfactant C is salt tolerant in all 3 brines, however, it did not provide low IFT required for oil recovery. Surfactants E, F and the mixture of surfactants as described in Test numbers 7-10 are salt tolerant and also provides low IFT necessary for oil recovery. The data in Table III demonstrated the unexpected low IFT and brine tolerance by using surfactants containing a weak anionic functionality group and a strong anionic functionality.

The ratio of the surfactant with strong anionic functionality group and the surfactant with weak anionic functionality group can be further optimized by varying their ratio based on the crude oil and brine properties as demonstrated in Table IV.

TABLE IV
Effect of IFT On Various Ratios of Surfactants D and E in BRINE 3
Crude Oil API Gravity = 27
Temperature = 65° C.

| Surfactant D, % | Surfactant E, % | IFT, mN/m |
|---|---|---|
| 100 | 0 | 0.891 |
| 75 | 25 | 0.0080 |
| 50 | 50 | 0.0058 |
| 25 | 75 | 0.0023 |
| 0 | 100 | 0.356 |

Table IV shows that surfactants D and E did not work effectively by themselves. By mixing surfactant D and E at various ratios, the optimum ratios for the crude oil and brine tested was 25% surfactant D and 75% surfactant E.

Based on the results revealed above in Tests 1-10 a composition has been developed for the recovery of oil from subterranean reservoirs by injecting an aqueous fluid into one or more injection wells comprised of

a) one or more bi-functional surfactants having both sulfonate and carboxylate functionality,

b) one or more co-surfactants/solvents

c) optionally a viscosity control agent

d) optionally an alkali, and

e) an aqueous based solvent

The one or more bi-functional surfactants having both sulfonate and carboxylate functionally have the following formula:

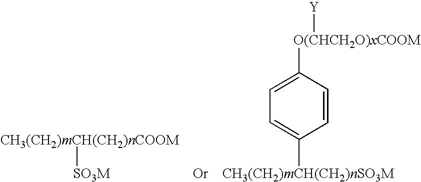

### Where m+n is 5 to 28, preferably 9 to 21 and most preferably 15 to 19,

### X is 0 to 30, preferably 0 to 15, and most preferably 4 to 12,

### Y is H, CH₃, or CH₃CH₂,

### M is any mono or divalent cations, preferably H, Na, K, Ca, Mg, NH₄, and most preferably Na.

A preferred formulation to inject into the reservoir is shown below: Surfactant concentrate: 0.05 to 2.0, preferably 0.05 to 0.5. and most preferably 0.1 to 0.3 wt % co-surfactants/solvents: 0 to 50 wt %, preferably 0 to 25 wt %, and most preferably 5 to 25 wt %, Viscosity improving agent: 0 to 10 wt %, preferably 0 to 3 wt %, and most preferably 0 to 0.5 wt %, Alkali: 0 to 10 wt %, preferably 0 to 5 wt %, and most preferably 0 to 2 wt %, and remainder is aqueous solvent,

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A composition for the recovery of oil from subterranean reservoirs comprised of
a) one or more surfactants having weak and strong anionic functionality groups,
b) an aqueous based solvent,
c) one or more co-surfactants/solvents,
d) optionally a viscosity increasing agent, and
e) optionally an alkali.

2. The composition for the recovery of oil as described in claim 1 where the one or more surfactants having weak and strong anionic functionality groups have the weak and strong functionality groups on the same molecule having one of the following structures: 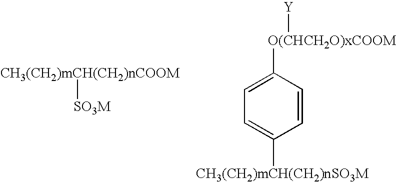
Where m+n is 5 to 28, preferably 9 to 21 and most preferably 15 to 19,
X is 0 to 30, preferably 0 to 15, and most preferably 4 to 12,
Y is H, CH₃, or CH₃CH₂,
M is a mono or divalent cation, preferably H, Na, K, Ca, Mg, NH₄, and most preferably Na.

3. The composition for the recovery of oil as described in claim 1 where the one or more surfactants having the weak and strong anionic functionality groups have these functionality groups contained on separate surfactants.

4. The composition for the recovery of oil having weak and strong anionic functionality groups on separate surfactants as described in claim 3 where the one or more surfactants having weak anionic functionality is chosen from the group carboxylated alkylphenol alkoxylates, carboxylated linear alcohol alkoxylates, carboxylated branched alcohol alkoxylates.

5. The composition for the recovery of oil having the weak and anionic functionality groups on separate surfactants as described in claim 3 where the one or more surfactants having strong anionic functionality is chosen from the group linear alkyl aryl sulfonates, branched alkyl aryl sulfonates, aryl linear alkyl sulfonates, aryl branched alkyl sulfonates, linear alkyl ether sulfates, branched alkyl ether sulfates, linear alkyl ether sulfonates, branched alkyl ether sulfonates, linear alkyl sulfates, branched alkyl sulfates, linear alkyl sulfonates, branched alkyl sulfonates.

6. The composition for the recovery of oil as described in claim 1 where the one or more anionic surfactants is present at a concentration from about 0.05% to about 2% by weight, preferably from about 0.05 to about 0.5% by weight, and most preferably from about 0.1% to about 0.3% by weight.

7. The composition for the recovery of oil described in claim 1 where the co-surfactants/solvent is present at a concentration from 0% to about 50% by weight, preferably from 0 to about 25% by weight, and most preferably from about 5% to about 25% by weight.

8. The composition for the recovery of oil described in claim 1 where the viscosity increasing agent is present at a concentration from 0 to about 10% by weight, preferably from 0 to about 3% by weight, and most preferably from 0 to about 0.5% by weight.

9. The composition for the recovery of oil described in claim 1 where the alkali is present at a concentration from 0 to about 10% by weight, preferably from 0 to about 5% by weight, and most preferably from 0 to about 2% by weight.

10. The composition for the recovery of oil described in claim 1 where the co-surfactants/solvents is chosen from the group of short chain alcohol, glycol, glycerin, glycol ether.

11. The composition for the recovery of oil described in claim 1 where the alkali is chosen from the group of hydroxide, carbonate, silicate, borate.

12. The composition for the recovery of oil described in claim 1 where the aqueous solvent is chosen from the group of water, synthetic brine, injection brine, produced brine.
